(19) United States
(12) Patent Application Publication
(10) Pub. No.: US 2010/0228103 A1
(43) Pub. Date: Sep. 9, 2010

(54) MULTIFACETED IMPLANTABLE SYNCOPE MONITOR - MISM

(75) Inventor: Stuart O. Schecter, Great Neck, NY (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/398,956

(22) Filed: Mar. 5, 2009

(51) Int. Cl. A61B 5/00

(52) U.S. Cl. 600/301

(57) ABSTRACT

A multi-channel implantable syncope monitor that monitors ECG data, myopotential data, EEG data, photoplethysmography (PPG) data, and position sensor data is used to capture physiologic data about a patient who is experiencing a syncopal event. The timing of the events within the simultaneously captured physiologic data can then be used to more accurately determine potential sources of origin of the syncopal event.

### FIELD OF THE INVENTION

The present invention relates to implantable monitoring devices and, in particular, concerns an implantable syncope monitoring device and methods of monitoring a plurality of different patient characteristics to determine possible causes or sources of syncope.

### BACKGROUND OF THE INVENTION

Syncope of unknown etiology is very common. A wide variety of different physiologic conditions can lead to syncope or fainting. These conditions can include orthostatic hypotension, vasovagal episodes, arrhythmic events that impede blood flow and cataplexy.

One difficulty that occurs with patients who suffer from syncope is that the cause of the syncope is often misdiagnosed and, thus, not effectively treated. For example, patients who suffer vasovagal episodes are often diagnosed as having epileptic episodes and are treated accordingly. Similarly, people suffering from epileptic episodes are often misdiagnosed as having vasovagal episodes.

One cause of the misdiagnosis of the cause of syncope and syncope-related events is that the implanted monitoring devices currently employed are not capable of measuring sufficient patient physiologic indicators that would allow for a more accurate diagnosis. For example, arrhythmia monitoring devices, such as those disclosed in U.S. Pat. No. 6,719,701 are capable of monitoring heart related factors such as electrocardiogram (ECG), heart rate, blood pressure, and body position. These factors allow for relatively accurate diagnosis of heart conditions that could lead to syncope events.

While these monitoring devices are effective at detecting physiologic conditions of the heart that could cause syncope related events, these devices are generally not capable of determining if the syncope related event is caused by epileptic sources or not. Indeed, cardiac-based monitoring devices are generally positioned within the body away from the patient's musculature so as to obtain ECG signals that are unaffected by the muscle contractions. This placement limits the ability of the device to sense physiologic characteristics of the muscles that may be indicative of a seizure related syncope.

Implanted cardiac-based monitoring devices also often lack the ability to sense photoplethysmography (PPG) data which limits the functionality of the monitoring device. PPG data can provide a more real time indication of the patient's hemodynamic and respiratory information, e.g., apnea, minute ventilation oxygenation, etc. Further, syncope monitoring systems are often not set up to capture a wide variety of signals simultaneously and are thus less capable of ascertaining temporal indications indicative of different sources of syncope-related events.

From the foregoing, it will be appreciated that there is a need for an improved implantable syncope monitoring system. More specifically, there is a need for a monitoring system that is capable of detecting physiologic conditions indicative of heart-based syncope events as well as physiologic conditions indicative of epileptic-based syncope events. There is a further need for a device that is capable of integrating PPG data into the analytic determination of the potential cause of syncope and a further need of an ability to capture multiple different channels of physiologic data in a manner that allows for temporal comparison.

### SUMMARY OF THE INVENTION

The aforementioned needs are satisfied by one exemplary embodiment of the present invention which includes an implantable syncope monitor that is capable of monitoring both cardiac related activities and myopotential activity that is associated with seizure events. The implantable monitor in this implementation preferably receives signals indicative of heart function and is further indicative of electrical impulses within the skeletal muscles that may indicate a seizure-based cause of the syncope. In one exemplary implementation, the implantable syncope monitor monitors both the patient's ECG signal via an implanted lead and further includes an electrode that is positioned so as to monitor the contractions within the patient's musculature such as, for example, the pectoral muscle.

In one further exemplary embodiment, the implantable syncope monitor is further equipped with a photoplethsymography (PPG) monitor that is capable of obtaining data indicative of the patient's hemodynamic and respiratory performance. The monitor is thus simultaneously receiving signals indicative of the patient's musculature contractions, the heart function and other hemodynamic and respiratory functions. As such, the monitor is better capable of capturing data indicative of the likely cause of syncope within the patient and is further better capable of illustrating the temporal relationship.

In further exemplary embodiments, further functionality, such as the ability to communicate with external EEG monitors, the ability to detect motion and orientation of the patient via accelerometers and the like, can further be implemented by the implantable syncope monitor to simultaneously receive additional data for determining the cause of syncope within a patient.

In one implementation, the implanted monitor is capable of simultaneously receiving different channels of data from different types of sensors. These can include cardiac signals, myopotential signals, PPG signals, EEG signals, body position signals or some combination thereof. By simultaneously receiving these different channels of data, the temporal relationship between physiologic characteristics of the patient, as evidenced by these channels of data, can be evaluated as a basis for determining potential sources of origin of syncope or syncope-related events.

By having an implanted monitor that monitors not just heart function but myopotentials and possibly hemodynamic and respiratory functions provides greater data acquisition for determining of the causes of syncope. In one exemplary implementation, the implanted monitor is configured to review the data and provide a diagnostic indication of the potential causes of observed syncope. In other implementations, the device determines when a syncope event is occurring and captures and stores data associated with the event for further downloading and evaluation by a treating medical professional. It will be appreciated that these and other objects and advantages of the present invention will become more apparent form the following description taken in conjunction with the accompanying drawings.

### BRIEF DESCRIPTION OF THE DRAWINGS

FIG. 1A is a schematic illustration of one exemplary embodiment of an implantable syncope monitor;

FIG. 1B is a block diagram of the implantable syncope monitor of FIG. 1A;

FIG. 2 is a flow chart that illustrates the basic operation of the implantable syncope monitor of FIGS. 1A and 1B;

FIGS. 3A-3C are illustrations of exemplary data curves received by the implantable monitor illustrating the diagnostic improvement stemming from the ability to capture multiple channels of physiologic data for the patient; and

FIG. 4 is a block diagram of one implementation of the implantable syncope monitor of FIGS. 1A and 1B wherein a diagnostic functionality is implemented.

### DETAILED DESCRIPTION OF THE PREFERRED EMBODIMENT

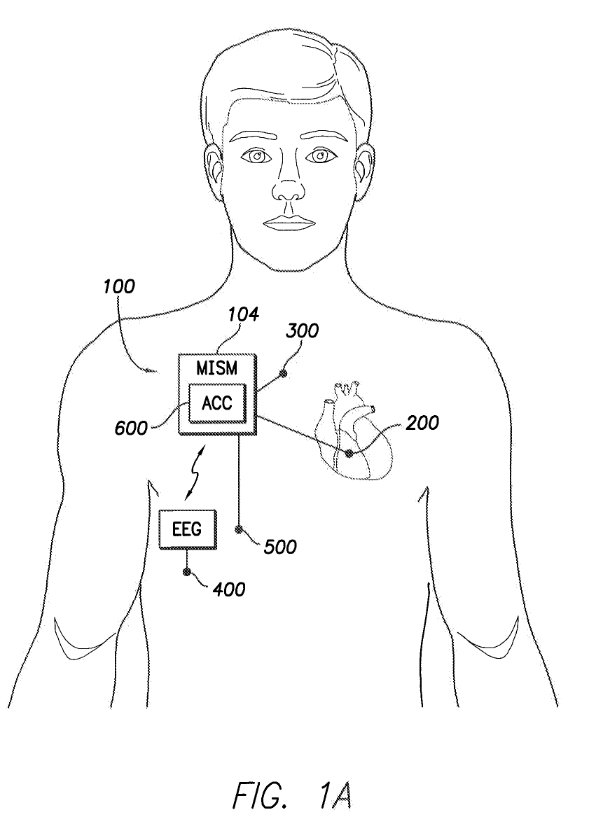

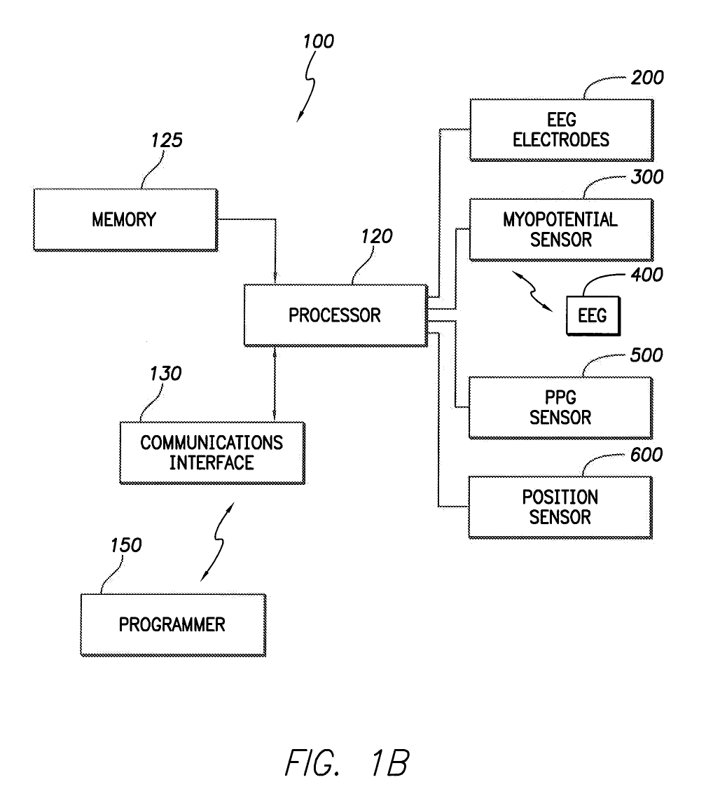

Reference will now be made to the drawings wherein like numerals refer to like parts throughout. Referring initially to FIGS. 1A and 1B, one embodiment of a multi-faceted implantable syncope monitor (MISM) 100 is shown. In this implementation, the MISM 100 is capable of monitoring multiple different types or channels of physiologic data about the patient, including heart related data, hemodynamic and respiratory related data and myopotential related data, simultaneously in order to be able to capture sufficient data to enable a more accurate diagnosis of the cause of syncope related events in a patient 102. Referring initially to FIG. 1A, the MISM 100 is shown implanted within the body of a patient. The actual implantation site can vary, depending upon circumstances, but one potentially efficacious implantation site is adjacent the pectoral muscle of the patient in a manner similar to the manner in which implantable cardiac stimulation devices are implanted. Indeed, the MISM 100 may actually be incorporated into the functionality of an implantable cardiac stimulation device such as a pacemaker, intra-cardioverter defibrillator or some device exhibiting the functionality of both without departing from the present technology.

As shown in FIG. 1A, the MISM 100 includes a myopotential sensor 300 that senses myopotential contractions within the skeletal muscles of the patient. In one specific implementation, the MISM 100 is contained within a casing or housing 104 of the MISM 100. In a standard pectoral implant procedure, the bottom surface of the housing 104 would be proximate the pectoral muscles and the myopotential sensors 300 are preferably positioned on the bottom surface so as to have greater access to the electrical signals indicative of the muscle contraction. In some implementations, the myopotential sensors 300 may be an EEG sensor 400 with band pass filtering that may be used to detect the myopotentials and to filter out EEG signal data.

As is also shown in FIG. 1A, the MISM 100 in this embodiment further includes electrodes 200 for sensing electrocardiogram (ECG) signals. The ECG signals can includes intra-cardio electrograms (IEG) signals that are obtained from leads that are implanted adjacent the heart or even within the chambers of the heart, such as leads associated with an implantable cardiac stimulation device. The ECG signals obtained by the electrodes 200 provide an indication of the electrical activity of the heart which can be used as a proxy for the performance of the heart or at least provide an indication of the onset of an arrhythmia that may be the source of a syncope related event.

In one implementation, the electrodes 200 are coupled to the housing 104 of the MISM 100 on the side distal from the pectoral muscles so that the pectoral muscle nerve activity less affects the sensing of the electrical activity of the heart by the electrodes 200. As is also shown in FIG. 1A, an external EEG monitor 400 can also be communicatively linked to the MISM 100 to provide signals indicative of the electrical performance of the heart or to provide a signal that can be filtered to obtain a myopotential signal in the manner discussed above.

The MISM 100 may also optionally include a photoplethysmography (PPG) sensor 500. PPG sensors 500 are optical-based sensors that sense hemodynamic data and respiratory data about the patient including blood oxygenation, blood flow, minute ventilation, etc. The PPG sensor 500 is implanted within the patient preferably at a location where data relating to blood flow adjacent the heart and respiratory function can be captured for subsequent evaluation. One such sensor is described in U.S. Pat. No. 6,719,701 entitled “Implantable Syncope Monitor and Method of Using Same” which is hereby incorporated by reference in its entirety.

In one implementation, the PPG sensor or sensors 500 can be located on the bottom side of the housing 104 of the MISM 100 and in another embodiment the PPG sensors 500 can be composed of fiber optic cables that direct red/infrared light towards the central vasculature. The PPG sensors derive a waveform characteristic of the arterial blood pressure. Band pass filtering can then be used to acquire PPG signals characteristic of arterial blood pressure, oxygenation and respiration.

As is also shown in FIG. 1A, a variety of other sensors can be included in the MISM 100. For example an accelerometer 600 can also be included which can be used to provide data indicative of the patient's activity level or of their posture. Activity level can provide an indication as to the origins of a syncope event as high activity levels in some patients can induce a respiratory or cardiac-based syncope. Further, an accelerometer 600 or similar device can also be configured to determine when a patient has suddenly changed orientations, e.g., has fallen down as a result of fainting, which can be useful for initiating data capture or determining potential causes of the syncope event.

FIG. 1B is an exemplary block diagram illustrating the functional components of the MISM 100. As shown, the MISM include a processor 120 that receives signals from the ECG electrodes 200, the myopotential electrodes 300, the EEG sensor 400, the PPG sensor 500, and the position sensor or accelerometer 600 in the manner described above. Further, the processor 120 is logically associated with one or more memories 125 that allow the processor 120 to store captured multiple channels of data indicative of the physiologic condition of the patient during syncope events.

The processor 120 is further able to communicate with a programmer device 150 in a well-known manner. The programmer 150 allows a treating medical professional to adjust the operational settings of the MISM 100 and further to download and receive the data that has been captured by the MISM 100 for further evaluation.

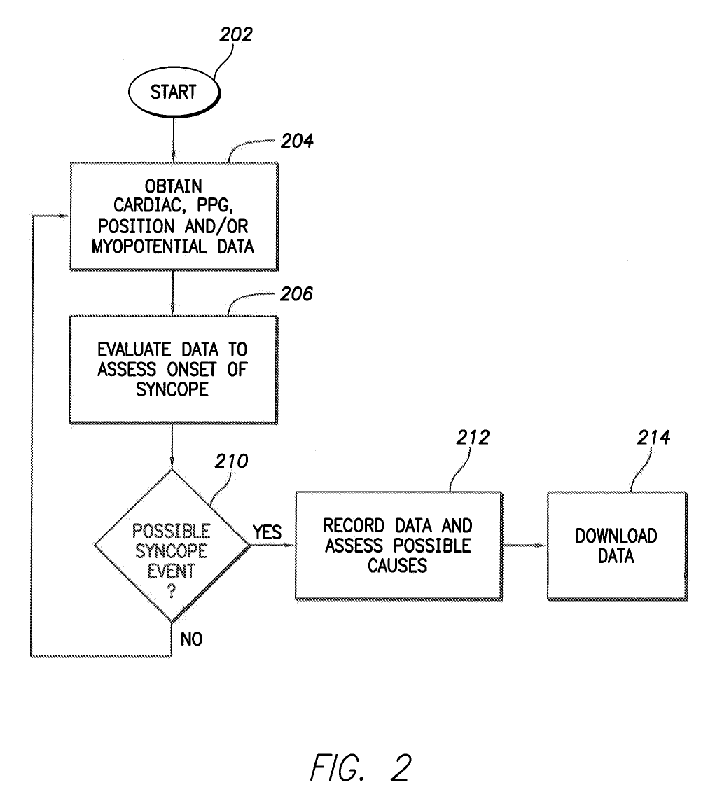

FIG. 2 is an exemplary flow chart that illustrates one manner in which the MISM 100 can operate to capture data to determine if the patient has suffered a syncope event and further to capture data to provide an indication of the source or cause of the syncope event. The flow chart of FIG. 2 is simply exemplary, the MISM 100 can be programmed to capture and evaluate data in any of a number of ways without departing from the spirit of the present invention.

As shown in FIG. 2, the MISM 100, from a start state 202 proceeds to capture data in state 204 from at least some of the implanted sensors 200, 300, 400, 500, and 600 in a known manner. Generally, the MISM 100 may be continuously receiving the data from the sensors or sampling the data on a periodic basis so as to conserve the battery power. Alternatively, the MISM 100 may be configured to monitor only a single sensor or a smaller group of sensors and when the single or smaller group of sensors provides data indicative of a syncope event, the MISM 100 may then enable the rest of the sensors.

The MISM 100 then evaluates the data from the sensors in state 206 to assess whether any of the sensors are indicating that there is a potential onset of a syncope related event. In one implementation, the MISM 100 is sampling all of the sensors 200-600 in state 204 and the MISM 100 has pre-recorded event indicators for each sensor, or for a combination of sensors, that are suggestive of a potential syncope event. For example, the MISM 100 may determine that there is a potential syncope event when the accelerometer 600 is indicating a sudden change in posture associated with the patient fainting. Further, the ECG sensor 200 or the EEG sensor 400 may also provide signals that correlate with cardiac arrhythmia or some other cardiac induced syncope. Similarly, the myopotential sensor 300 may also detect the activation of muscle cells that may be indicative of an epileptic episode that may also be a pre-cursor of a syncope event. As will be understood, the MISM 100 can be adapted to look for particular characteristic waveforms that may be indicative of syncope events and then use these indications to determine, in decision state 210, that a potential syncope-related event is occurring.

In the event that the MISM 100 determines that a potential syncope-related event is occurring, the MISM 100 is then adapted to record data sensed from some or all of the relevant sensors in state 212. In this way, multiple different signals from multiple different sensors can be simultaneously obtained during the onset of a potential syncope-related event. This information can either be used by the MISM 100 to ascertain a potential source of the syncope-related event or can be stored for subsequent download in state 214 to the programmer 150 for future evaluation by a treating medical professional. The MISM 100 can continue performing this monitoring and capturing of data relating to syncope-related events during the entire time of implantation. In this way, multiple potential events can have multiple channels of different data recorded to thereby allow for a more accurate diagnosis of the potential causes of the syncope events.

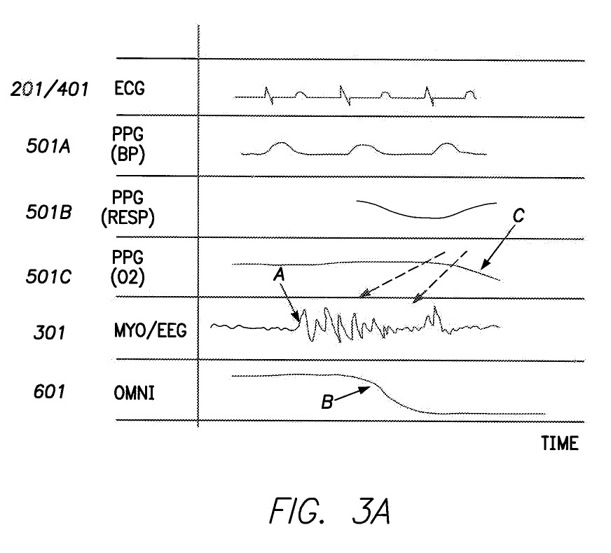

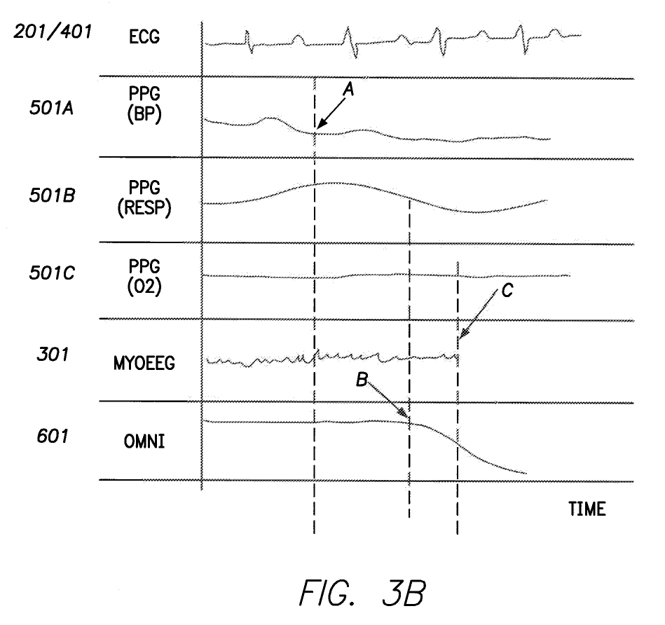

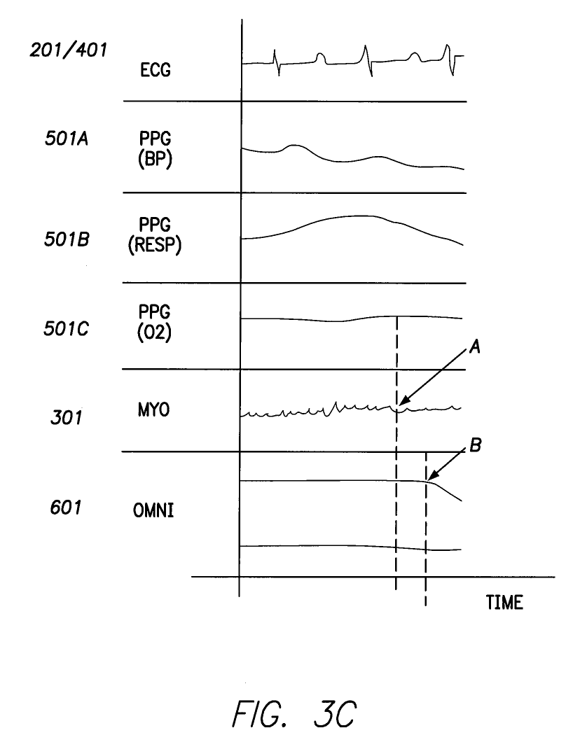

As discussed above, if only a single channel of analysis is used, e.g., only cardiac such as IEG signals or EEG signals, then non-cardiac based syncope events may be inaccurately diagnosed. Further, the temporal relationship between different sensed physiologic parameters may also provide an indication of potential sources of syncope-related events. FIGS. 3A through 3C provide examples of different events where a more accurate diagnosis can be obtained as a result of simultaneous capture of heart signals, myopotential signals, PPG signals, acceleration signals and the like.

Specifically, in FIG. 3A the patient incurs a syncopal episode. From an ECG/EEG 201/401 signal, the patient's cardiac rhythm is normal. From PPG signals 501a, 501b, the patient's blood pressure and respiration rate are also normal. However, from the PPG signal 501c, there is an indication that the oxygen saturation is normal, at initially 95%, but begins to decline towards the tail end of the monitored strip (arrow C). At the same time, at the myopotential channel 301, there is observed the beginning of erratic, chaotic waveforms as represented by the dotted arrows in FIG. 3A, that are representative of myopotentials indicating spontaneous tonic-clonic muscle contractions and/or ambulatory EEG detection of ictus via communication between an ambulatory EEG monitor and the MISM 100.

In this specific example, the MISM 100 is preferably capable of distinguishing between myopotentials characteristic of active skeletal muscle contractions and that due to various types of seizure activity. Here, seizure activity may be characterized as tonic-clonic contractions of skeletal muscles for 10-30 seconds with a characteristic pattern. Other pattern characteristics may also be identified and the MISM 100 may be further programmed to recognize these other characteristic patterns, e.g., tonic, myclonic, clonic, atonic, or absence (petit mal).

As is further illustrated in FIG. 3A, an accelerometer signal 601 may also provide an indication that, following the onset of the myopotentials, the patient may fall as noted by arrow B. Further, the oxygen saturation begins to fall, as indicated by arrow C which indicates that the patient has suffered an epileptic-based syncopal episode.

In this implementation, the myopotential sensor 300 acquires myopotential data 301 alone. It will be appreciated that, in alternative embodiments, the myopotential sensor 300 may acquire both myopotential and EEG data. Simultaneously obtaining this data may help differentiate between various forms of ictus. For example, by simultaneously capturing EEG and myopotential data, evidence of a seizure without tonic-clonic muscle contraction may be sensed. This scenario may be indicative of a petit mal or absence seizure which responds to different pharmacologic agents than grand mal seizures.

Pseudoseizures are an example of seizure activity that is not physiologically mediated and often elude diagnosis. The finding of typified myopotentials (e.g., not tonic-clonic) in the absence of EEG evidence of seizure activity or other physiologic abnormalities would be consistent with pseudoseizures and direct the clinician to send the patient for psychiatric counseling. This underscores the value of sensing multiple different physiologic channels simultaneously in an effort to diagnose a source of ictus such as syncope.

In FIG. 3B the ECG signal 201 is normal and is not indicative of any arrhythmia. However, the PPG monitor 501a detects a blood pressure drop as the initial event (arrow A) but the PPG monitors for oxygen saturation 501b and respiration 501c are initially unaffected. The myopotential/EEG signal 301 is, however, detecting tonic-clonic activity (arrow C) at about the same time the accelerometer signal 601 is indicating that the patient has fallen (arrow B). These signals are indicative of a hypotensive episode that leads to a loss of balance and a subsequent seizure from cerebral hypoperfusion. In this case, systematic oxygenation is maintained, as is evidenced by the sensor 301b, and the drop in blood pressure is not secondary to arrhythmia or change in body position such as that caused by orthostasis.

The combined PPG sensing and ECG/myopotential sensing reveals the correct diagnosis as dysautonomia with a strong vasodepressor component. A traditional implantable syncope monitor would not review an etiology and if the episode were witnessed, it is possible it would have been misdiagnosed as a primary seizure which may lead to incorrect therapy being prescribed for the patient.

FIG. 3C provides signals from the multiple sensors that are indicative of the patient having a drop attack. In this scenario, the cardiac signal 201, 401, the blood pressure 501a, the respiration 501b and the oxygen saturation 501c are all normal. However, the position sensor 601 indicates that the patient has suffered a fall (arrow B) that is occurring after the change in myopotential sensor data 301 that is indicative of a loss of muscle tone (arrow A). This scenario is consistent with cataplexy which is an under-diagnosed syndrome given the complexity of the causative physiologic processes and is easily confused with other syndromes such as vasovagal or absence seizures. In this example, there is a loss of muscle tone just prior to the fall without seizure activity or change the vital signs as evidenced by the cardiac or PPG signals.

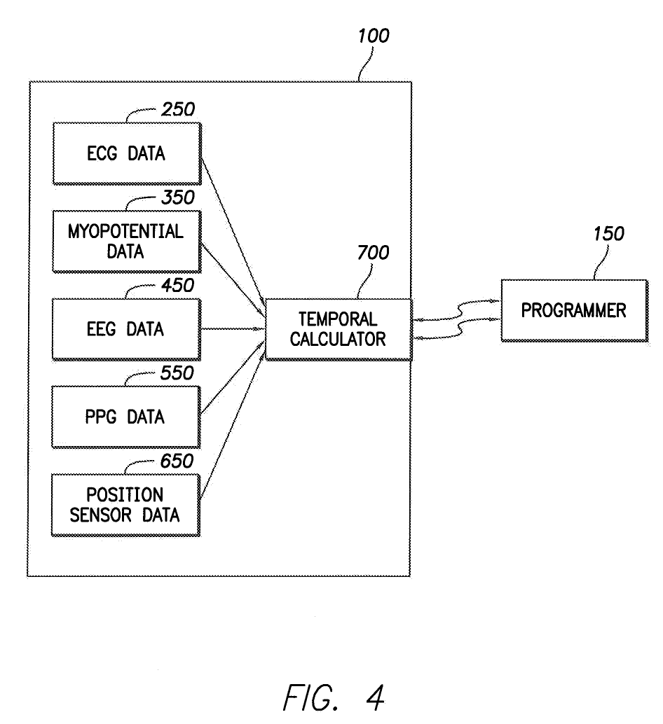

As is illustrated in the examples of FIGS. 3A-3C, the simultaneous capture of the different channels of data allows for a temporal comparison of each of the channels of data which can be used for diagnostic purposes. The temporal occurrence of different physical characteristics in the patient can often be used to diagnose the potential source of origin of a syncopal event. In one implementation, as shown in FIG. 4, ECG data 250, the mypotential data 350, EEG data 450, PPG data 550 and position sensor data 650 are all fed simultaneously into a temporal calculator 700 that is functionally implemented by the processor 120 of the MISM 100. Preferably, in this implementation, the temporal calculator 700 evaluates the signals from the various sensors and makes a preliminary diagnosis of the cause of the syncopal event which can then be output to the MISM programmer 150 in a known manner. Alternatively, the stored data can simply be downloaded to the programmer 150 to allow the treating medical personnel to perform their own diagnosis.

While the foregoing description has shown, illustrated and described the fundamental novel features of the present teachings, it will be apparent that various omissions, substitutions and changes to the form the detail of the apparatus as illustrated, as well as the uses thereof, may be made by those of ordinary skill in the art without departing from the scope of the present teachings. Hence, the scope of the present teachings should not be limited to the foregoing discussion, but should be defined by the appended claims.

What is claimed is:

1. An implantable system for capturing data about potential syncope events, the system comprising:
a cardiac sensor that captures signals from the patient's body indicative of the function of the patient's heart;
a seizure sensor that captures signals from the patient's body indicative of potential seizure activity of the patient;
a hemodynamic sensor that captures signals from the patient's body indicative of the hemodynamic performance of the patient's body; and
a processor that receives the signals from the cardiac sensor, the seizure sensor and the hemodynamic sensor, so that the signals detected by the cardiac sensor, the seizure sensor and the hemodynamic sensor can be evaluated to assess potential causes of syncope events.

2. The system of claim 1, wherein the cardiac sensor comprises a sensor that receives ECG signals from leads positioned proximate the heart of the patient.

3. The system of claim 1, wherein the cardiac sensor comprises an EEG sensor that is positioned about the body of the patient.

4. The system of claim 1, wherein the hemodynamic sensor provides an indication of respiration, blood pressure and oxygen saturation.

5. The system of claim 4, wherein the hemodynamic sensor includes a photoplesthysmography (PPG) sensor that optically captures signals from the blood of the patient.

6. The system of claim 1, further comprising an accelerometer-based sensor that senses the movement of the patient.

7. The system of claim 6, wherein the accelerometer-based sensor detects when the patient has changed orientation and thereby provides an indication of whether the patient has fainted.

8. The system of claim 1, wherein the seizure sensor comprises a myopotential sensor that senses skeletal muscles contractions of the patient.

9. The system of claim 1, wherein the processor is adapted to identify indicia within the received signals that are indicative of a potential syncope-related event.

10. The system of claim 9, wherein the processor is adapted to store data received from the sensors when the programmer has identified indicia in the received signals that are indicative of the potential syncope-related event.

11. The system of claim 9, wherein the processor is adapted to analyze the received signals to ascertain a potential source of origin of the syncope-related event.

12. An implantable system for capturing data about potential syncope events, the system comprising:
a plurality of sensors that sense a plurality of different physiologic characteristics of the patient, including physiologic characteristics indicative of cardiac performance, seizure activity and hemodynamic function; and
a processor that simultaneously receives signals from the plurality of sensors wherein the processor evaluates the signals from the plurality of sensors so that the relative timing of events detected by the plurality of sensors can be used to ascertain possible sources of potential syncope events.

13. The system of claim 12, wherein the plurality of sensors include a cardiac sensor which comprises a sensor that receives ECG signals from leads positioned proximate the heart of the patient.

14. The system of claim 13, wherein the cardiac sensor comprises an EEG sensor that is positioned about the body of the patient.

15. The system of claim 12, wherein the plurality of sensors include a hemodynamic sensor which provides an indication of respiration, blood pressure and oxygen saturation.

16. The system of claim 15, wherein the hemodynamic sensor includes a photoplesthysmography (PPG) sensor that optically captures signals from the blood of the patient.

17. The system of claim 12, wherein the plurality of sensors include an accelerometer-based sensor that senses the movement of the patient.

18. The system of claim 17, wherein the accelerometer-based sensor detects when the patient has changed orientation and thereby provides an indication of whether the patient has fainted.

19. The system of claim 12, wherein the plurality of sensors include a seizure sensor that comprises a myopotential sensor that senses skeletal muscles contractions of the patient.

20. The system of claim 12, wherein the processor is adapted to identify indicia within the received signals that are indicative of a potential syncope-related event.

21. The system of claim 20, wherein the processor is adapted to store data received from the sensors when the programmer has identified indicia in the received signals that are indicative of the potential syncope-related event.

22. The system of claim 20, wherein the processor is adapted to analyze the received signals to ascertain a potential source of origin of the syncope-related event.

23. A method of monitoring physiological signals relating to potential sources of syncope-related events, the method comprising:
implanting a plurality of sensors within the body of a patient that monitor a plurality of different physiologic parameters including hemodynamic status, heart rate and seizure activity;
simultaneously capturing signals from the plurality of sensors; and
comparing the relative timing of events in the captured signals to ascertain potential sources of syncope-related events.

24. The method of claim 23, wherein the plurality of sensors include a cardiac sensor which comprises a sensor that receives ECG signals from leads positioned proximate the heart of the patient.

25. The method of claim 24, wherein the cardiac sensor comprises an EEG sensor that is positioned about the body of the patient.

26. The method of claim 23, wherein the plurality of sensors include a hemodynamic sensor which provides an indication of respiration, blood pressure and oxygen saturation.

27. The method of claim 26, wherein the hemodynamic sensor includes a photoplesthysmography (PPG) sensor that optically captures signals from the blood of the patient.

28. The method of claim 23, wherein the plurality of sensors include an accelerometer-based sensor that senses the movement of the patient.

29. The method of claim 28, wherein the accelerometer-based sensor detects when the patient has changed orientation and thereby provides an indication of whether the patient has fainted.

30. The method of claim 23, wherein the plurality of sensors include a seizure sensor that comprises a myopotential sensor that senses skeletal muscles contractions of the patient.
